(19) United States
(12) Patent Application Publication
(10) Pub. No.: US 2025/0066829 A1
(43) Pub. Date: Feb. 27, 2025

(54) OZONE TREATMENT OF FERMENTATION MEDIA

(71) Applicant: LOCUS SOLUTIONS IPCO, LLC, SOLON, OH (US)

(72) Inventors: LEE SPEIGHT, SOLON, OH (US); NICHOLAS CALLOW, SOLON, OH (US); TYLER DIXON, MADISON, OH (US)

(21) Appl. No.: 18/258,360

(22) PCT Filed: May 10, 2023
(86) PCT No.: PCT/US2023/066814

§ 371 Date: Jun. 20, 2023

Related U.S. Application Data
(60) Provisional application No. 63/342,423, filed on May 16, 2022.

(51) Int. Cl. C12P 19/44; A61L 2/20; C12M 1/00; C12M 1/12

(52) U.S. Cl. CPC C12P 19/44; A61L 2/202; C12M 29/06; C12M 37/06

(57) ABSTRACT

The subject invention provides methods for enhanced production of microbial biosurfactants using a fermentation medium that has been sterilized using ozone. The methods provide for higher yields of the biosurfactants, improved color, and reduced energy expenditure compared with standard fermentation techniques that rely upon thermal sterilization.

### CROSS-REFERENCE TO RELATED APPLICATION

This application claims priority to U.S. Provisional Patent Application No. 63/342,423, filed May 16, 2022, which is incorporated herein by reference in its entirety.

### BACKGROUND OF THE INVENTION

Biosurfactants are a structurally diverse group of surface-active substances produced by microorganisms. All biosurfactants are amphiphiles. They consist of two parts: a polar (hydrophilic) moiety and non-polar (hydrophobic) group. Due to their amphiphilic structure, biosurfactants can, for example, increase the surface area of hydrophobic water-insoluble substances, increase the water bioavailability of such substances, and change the properties of microbial cell surfaces. Biosurfactants can accumulate at interfaces, thus reducing interfacial tension and leading to the formation of aggregated micellar structures in solution. The formation of micelles provides a physical mechanism to mobilize, for example, oil in a moving aqueous phase.

There are multiple types of biosurfactants, including low molecular weight glycolipids, lipopeptides, flavolipids and phospholipids, and high molecular weight polymers such as lipoproteins, lipopolysaccharide-protein complexes, and polysaccharide-protein-fatty acid complexes.

Specifically, glycolipids are biosurfactants comprising a carbohydrate and at least one fatty acid. Glycolipids include, for example, rhamnolipids (RLP), rhamnose-d-phospholipids, trehalose lipids, trehalose dimycolates, trehalose monomycolates, mannosylerythritol lipids (MEL), cellobiose lipids, ustilagic acids and sophorolipids (SIP).

Fermentation of certain microbial cells in a culture substrate including a sugar and/or lipids and fatty acids with carbon chains of differing length can be used to produce a variety of glycolipids. When fermenting any microorganism, however, there are several important considerations involved in obtaining high quantities of high-quality end products, including, for example, the components of the fermentation medium, the risk of contamination and the purity of the end products.

Contamination, in particular, can result from the presence of undesirable microorganisms in the water, nutrients and/or equipment utilized during fermentation. The addition of antimicrobial compounds into a fermentation medium before and/or after cultivation is one way to control contaminating microbes within a culture; however, these additional components must not negatively-affect the intended microbial inoculant. Some antimicrobials are harsh chemicals that require greater care when handling. Furthermore, the use of substances characterized as antibiotics can be undesirable due to the possibility of facilitating antibiotic resistance.

Other methods for preventing contamination include, for example, sterilizing the components of the liquid fermentation medium by pasteurization, steaming or autoclaving. While effective, these methods unfortunately require large amounts of energy to provide the necessary level of heat. Furthermore, the use of heat in fermentation media containing nitrogen and sugars will often facilitate the production of undesirable side-products within a fermentation end product, such as Maillard reaction products. These can adversely affect the color and odor of the end product, leading to increased need for purification.

Biosurfactants have the potential to be useful in a wide variety of industries, including home care, personal care, oil and gas recovery and agriculture. Thus, the large-scale production of biosurfactants is necessary for the efficient and cost-effective development of such products. One potential route through which increased efficiency and scale can be achieved is through improved methods of sterilizing fermentation media.

### BRIEF SUMMARY OF THE INVENTION

The subject invention provides methods for enhanced production of microbial metabolites, as well as improved compositions produced according to these methods.

In certain specific embodiments, the subject invention provides for the sterilization of aerobic fermentation media and/or media components by applying ozone to the media and/or media components.

Advantageously, the subject methods reduce the energy expenditure of sterilization compared with standard methods, while providing unexpected additional benefits to quality and productivity of end-products. Furthermore, the subject invention is suitable for industrial scale production of microbial metabolites and uses safe and environmentally friendly materials and processes.

In certain embodiments, the subject invention provides methods of enhancing a microbial fermentation process, wherein the methods comprise applying ozone to the fermentation media and/or components thereof. Preferably, the fermentation process is an aerobic fermentation process. Even more preferably, the fermentation process is an aerobic fermentation process for the production of biosurfactants.

In certain embodiments, the methods of the subject invention enhance the microbial fermentation process by sterilizing the fermentation media. Advantageously, in certain embodiments, the methods can be used to replace thermal and energy-intensive methods of sterilization, such as pasteurization, steaming and/or autoclaving.

In certain embodiments, the methods enhance the microbial fermentation process by, surprisingly, increasing microbial productivity and decreasing impurities levels in the finished product, e.g., the biosurfactant. In some embodiments, the use of ozone as a sterilizing agent in the fermentation medium can improve oxygen transfer during aerobic fermentation, which can, for example, improve the ability to scale up fermentation to industrial-scale levels and/or increase cell growth and/or productivity.

In some embodiments, an additional unknown mechanism of action is occurring that leads to a surprising increase in the quantity of metabolites and growth by-products produced by a microorganism, including biosurfactants.

In some embodiments, the use of ozone significantly reduces fermentation side-products, such as, for example, Maillard reaction products resulting from the coupling of nitrogenous species and sugars that can result from high-temperature techniques. This not only improves the color and/or odor of the end-product, but also provides a method for sterilizing fermentation media containing nitrogen and sugar components that can result in undesirable color- and/or odor-causing impurities.

In certain specific embodiments, the subject invention provides enhanced methods of fermenting microorganisms, wherein the methods comprise obtaining media components suitable for aerobic fermentation of a microorganism and placing the components, either individually or together, into a vessel. Preferably, the microorganism is a biosurfactant-producing microorganism, and the media components are appropriate for facilitating the production of said biosurfactants. In some embodiments, the vessel is a fermentation tank into which the microorganism will be inoculated and ultimately cultivated.

In certain embodiments, the method comprises applying the ozone to the vessel such that it is contacted with the media components. In some embodiments, the ozone can be supplied via, for example, an ozone generator or ozone machine coupled directly to the vessel, which delivers the ozone to the inside of the vessel via, for example, air spargers. In certain embodiments, the ozone supply is coupled to the vessel and/or the spargers via piping.

The ozone can be applied continuously for a period of time sufficient to achieve sterilization of the fermentation medium within the vessel, for example, from 5 minutes to 48 hours, from 30 minutes to 36 hours, from 60 minutes to 24 hours, or from 90 minutes to 12 hours.

In some embodiments, the method can be carried out at ambient temperature, without heating or cooling of the ozone and/or fermentation media components. In some embodiments, the ozone can be cooled to, e.g., 5 to 15° C. prior to contact with the fermentation medium using standard cooling techniques.

In certain embodiments, the fermentation medium is agitated throughout the process of being contacted with the ozone. In some embodiments, this is achieved through use of air spargers or another mechanism for producing bubbles. In some embodiments, this is achieved through mechanical mixing, such as with an impeller or other manual or automatic mixing device.

In certain embodiments, the fermentation medium can be tested for contamination after ozone sterilization and prior to inoculation of the medium with a microorganism. If testing reveals that sterilization has not been achieved, then additional ozone treatments can be applied.

Once sterilization of the fermentation medium is achieved according to the subject methods, the medium can be inoculated with a microorganism of choice to proceed with aerobic fermentation. In preferred embodiments, the microorganism is a yeast, fungus or bacterium capable of producing a biosurfactant, including, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes.

In certain preferred embodiments, the biosurfactant is a glycolipid, even more preferably a sophorolipid (SLP). In certain embodiments, the sophorolipid-producing microorganism is Starmerella bombicola, or another member of the Starmerella and/or Candida clades. For example, S. bombicola strain ATCC 22214 can be used according to the subject methods. In certain embodiments, S. bombicola can surprisingly tolerate and/or even thrive in the presence of peroxide in the fermentation medium, which results from the decomposition of ozone.

In preferred embodiments, the methods of the subject invention comprise cultivating a sophorolipid-producing yeast in a submerged fermentation reactor to produce a yeast culture, said yeast culture comprising liquid fermentation broth, yeast cells and a mixture of SLP molecules; extracting the SLP from the yeast culture; and, optionally, purifying the SLP. In some embodiments, the methods comprise testing the extracted and/or purified SLP for impurities, color and/or odor.

Advantageously, the present invention can be used without causing harm to users and without requiring high energy inputs or releasing large quantities of polluting and/or toxic compounds into the environment. Additionally, the compositions and methods utilize components that are biodegradable and toxicologically safe.

### DETAILED DESCRIPTION OF THE INVENTION

The subject invention provides novel methods for sterilizing fermentation media and/or media components used for aerobic fermentation of microorganisms, wherein the methods comprise applying ozone to the media and/or media components. Advantageously, the subject methods reduce the energy expenditure of sterilization compared with standard methods, while providing unexpected additional benefits to quality and productivity. Furthermore, the subject invention is suitable for industrial scale production of microbial metabolites and uses safe and environmentally friendly materials and processes.

### Selected Definitions

In certain embodiments, the subject invention provides improved methods for fermenting glycolipid biosurfactants, such as, for example, sophorolipids. Sophorolipids are glycolipid biosurfactants produced by, for example, various yeasts of the Starmerella clade. SLP consist of a disaccharide sophorose linked to long-chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid can have, 15 for example, 11 to 20 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form). In most cases, fermentation of SLP results in a mixture of hydrophobic (water-insoluble) SLP, including, e.g., lactonic SLP, mono-acetylated linear SLP and di-acetylated linear SLP, and hydrophilic (water-soluble) SLP, including, e.g., non-acetylated linear SLP.

As used herein, the term “sophorolipid,” “sophorolipid molecule,” “SLP” or “SLP molecule” includes all forms, and isomers thereof, of SLP molecules, including, for example, acidic (linear) SLP and lactonic SLP. Further included are mono-acetylated SLP, di-acetylated SLP, esterified SLP, SLP with varying hydrophobic chain lengths, SLP with fatty acid-amino acid complexes attached, and other, including those that are and/or are not described within in this disclosure.

In some embodiments, the SLP molecules according to the subject invention are represented by General Formula (1) and/or General Formula (2) and are obtained as a collection of 30 or more types of structural homologues having different fatty acid chain lengths (R³), and, in some instances, having an acetylation or protonation at R¹ and/or R².

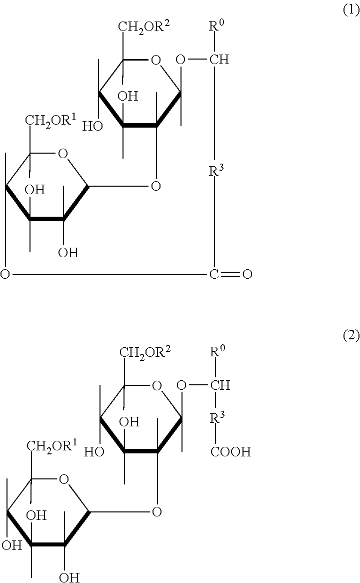

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more Substituents.

Non-limiting examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, and others. R³ typically has up to 20 carbon atoms.

As used herein, a “biofilm” is a complex aggregate of microorganisms, such as bacteria, yeast, or fungi, wherein the cells adhere to each other and/or to a surface using an extracellular matrix. The cells in biofilms are physiologically distinct from planktonic cells of the same organism, which are single cells that can float or swim in liquid medium.

As used herein, “contaminant” refers to any substance that causes another substance or object to become fouled, contaminated or impure. Contaminants can be living or non-living and can be inorganic or organic substances or deposits. Furthermore, contaminants can include, but are not limited to, hydrocarbons, such as petroleum or asphaltenes; fats, oils and greases (FOG), such as cooking grease, plant-based oils, and lard; lipids; waxes, such as paraffin; resins; microorganisms, such as bacteria, biofilms, viruses, fungi, molds, mildews, protozoa, parasites or another infectious microorganisms; stains; or any other substances referred to as, for example, dirt, dust, scale, sludge, crud, slag, grime, scum, plaque, buildup, or residue.

As used herein, “fouling” or “contamination” mean the presence, accumulation or deposition of contaminants on a surface or in an environment, e.g., a fermentation vessel, in such a way as to compromise the structural and/or functional integrity of the surface or environment.

As used herein, “cleaning” as used in the context of contaminants or fouling means removal or reduction of contaminants from a material and/or surface.

As used herein, “control” in the context of a microorganism means killing, immobilizing, destroying, removing, reducing population numbers of, and/or otherwise rendering the microorganism incapable of reproducing and/or causing substantial harm or fouling. In preferred embodiments, the deleterious microorganisms are “substantially controlled,” meaning at least 90%, preferably at least 95%, or more preferably, at least 99% of the microorganism's population within a specified area is controlled. In certain preferred embodiments, 100% of the deleterious microorganism is controlled, meaning the surface and/or material has been “sanitized” or “sterilized.”

As used herein, to “disinfect” means to control or substantially control a deleterious microorganism in 10 minutes or less, preferably in 5 minutes or less, more preferably in 2 minutes or less, after the time of contact between the composition and the deleterious microorganism (i.e., exposure time).

As used herein, a “deleterious” or “pathogenic” microorganism refers to any single-celled or acellular organism that is capable of causing an infection, disease or other form of harm in another organism, or to cause fouling in a fermentation process. As used herein, deleterious or pathogenic microorganisms can include, for example, bacteria, cyanobacteria, biofilms, viruses, virions, viroids, fungi, molds, mildews, protozoa, prions, and algae. In certain embodiments, a deleterious microorganism can include multicellular organisms, such as, for example, certain parasites, helminths, nematodes and/or lichens. Non-limiting examples of fermentation contaminants include Lactobacillus spp., Dekkera spp., Fusarium spp., Pediococcus spp., and certain bacteriophages.

As used herein “fermentation” refers to growth or cultivation of cells under controlled conditions. The growth could be aerobic or anaerobic, although in preferred embodiments, the growth is aerobic. The cultivation can be characterized as batch, quasi-continuous, or continuous cultivation. Unless the context requires otherwise, the phrase is intended to encompass both the growth phase and product biosynthesis phase of the process.

As used herein, a “broth,” “culture broth,” or “fermentation broth” refers to a culture medium comprising at least nutrients. If the broth is referred to after a fermentation process, the broth may comprise microbial growth byproducts and/or microbial cells as well.

As used herein, “preventing” a situation or occurrence refers to avoiding, delaying, forestalling, or minimizing the onset of a particular sign or symptom of situation or occurrence. Prevention can, but is not required to be, absolute or complete, meaning the situation or occurrence may still develop at a later time. Prevention can include reducing the severity of the onset of situation or occurrence, and/or inhibiting the progression of the situation or occurrence to one that is more severe.

As used herein, “surfactant” refers to a substance or compound that reduces surface tension when dissolved in water or water solutions, or that reduces interfacial tension between two liquids, or between a liquid and a solid. The term “surfactant” thus includes cationic, anionic, nonionic, zwitterionic, amphoteric agents and/or combinations thereof. By “biosurfactant” is meant a surfactant produced by a living cell and/or using naturally-derived sources.

As used herein, an “isolated” or “purified” nucleic acid molecule, polynucleotide, polypeptide, protein or organic compound, such as a small molecule, is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of other molecules, or the amino acids that flank it, in its naturally-occurring state. An “isolated” strain means that the strain is removed from the environment in which it exists in nature. Thus, the isolated strain may exist as, for example, a biologically pure culture, or as spores (or other forms of the strain).

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end-product of metabolism. Examples of metabolites include, but are not limited to, enzymes, acids, solvents, alcohols, proteins, vitamins, minerals, microelements, amino acids, biopolymers and biosurfactants.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduces” means a negative alteration, and “increases” means a positive alteration, wherein the alteration is at least 0.001%, 0.01%, 0.1%, 1%, 5%, 10%, 15%, 20%, 25%, 30%, 35%, 45%, 50%, 55%, 60%, 65%, 70%, 75%, 80%, 85%, 90%, 95%, 99%, or 100%, inclusive of all values therebetween.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. All references cited herein are hereby incorporated by reference.

### Methods of Fermentation

The subject invention provides enhanced methods of fermenting microorganisms with reduced energy expenditure, reduced impurities, and/or improved cell and/or metabolite productivity.

In preferred embodiments, methods are provided for treating fermentation media and/or media components to enhance a microbial fermentation process, wherein the methods comprise applying ozone to the fermentation media and/or components. Preferably, the fermentation process is an aerobic fermentation process. More preferably, the fermentation process is an aerobic fermentation process for the purpose of producing biosurfactants.

In certain embodiments, the methods enhance the microbial fermentation process by sterilizing the fermentation media. Advantageously, in certain embodiments, the methods can be used to replace thermal and energy-intensive methods of sterilization, such as pasteurization, steaming and/or autoclaving.

In certain embodiments, the methods enhance the microbial fermentation process by, surprisingly, increasing productivity of the microorganisms and decreasing impurities levels in the end-product, e.g., the biosurfactant. In some embodiments, the application of ozone as to the fermentation medium improves oxygen transfer during aerobic fermentation, which can, for example, improve the ability to scale up fermentation to industrial-scale levels and/or increase cell growth and/or productivity. In some embodiments, the method leads to a surprising increase in the quantity of metabolites and growth by-products produced by a microorganism, including biosurfactants.

In some embodiments, the use of ozone, a nonthermal sterilizing agent, significantly reduces fermentation side-products, such as, for example, Maillard reaction products resulting from the coupling of nitrogenous species and sugars. This not only improves the color and/or odor of the end-product, but also provides a method for sterilizing fermentation media containing nitrogen and sugar components that would otherwise result in undesirable browning and/or odor-causing impurities.

In certain specific embodiments, the subject invention provides enhanced methods of fermenting microorganisms, wherein the methods comprise obtaining media components suitable for aerobic fermentation of a microorganism and placing the components, either individually or together, into a vessel.

The vessel used according to the subject invention can be any container, fermenter or cultivation reactor. As used herein, the term “reactor,” “bioreactor,” “fermentation reactor” or “fermentation vessel” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments, the bioreactor may comprise a first growth reactor and a second fermentation reactor. As such, when referring to the addition of substrate to the bioreactor or fermentation reaction, it should be understood to include addition to either or both of these reactors where appropriate.

The vessel can range in volume from a few gallons to thousands of gallons. In some embodiments, the vessel can hold about 1 gallon to about 1,500 gallons, or about 1 L to about 6000 L. In some embodiments, a plurality of vessels can be set up inside an enclosure or housing facility to produce even greater total volumes of one or more fermentation products.

In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, metabolite concentration, and/or visible oil level monitoring.

In certain embodiments, the fermentation medium or medium components that are placed in the vessel are in liquid form or are mixed with water to form a liquid. Preferably, the media components are appropriate for facilitating the production of biosurfactants via aerobic fermentation.

In certain embodiments, the fermentation medium comprises one or more sources of carbon. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, madhuca oil, soybean oil, rice bran oil, olive oil, corn oil, sunflower oil, sesame oil, and/or linseed oil; powdered molasses, etc. These carbon sources may be used independently or in a combination of two or more.

In certain embodiments, the fermentation medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In certain embodiments, the fermentation medium comprises one or more inorganic salts. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In certain embodiments, the fermentation medium comprises growth factors and/or trace nutrients for microorganisms. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

In certain embodiments, upon obtaining the fermentation medium or component thereof and placing it in the vessel, the method comprises applying the ozone to the vessel such that it is contacted with the medium. In some embodiments, the ozone can be supplied via, for example, an ozone generator or ozone machine coupled directly to the vessel, which delivers the ozone to the inside of the vessel.

In some embodiments, the ozone generator or ozone machine is coupled to a sparging system. In certain embodiments, the sparging system comprises stainless steel injectors that produce microbubbles. In an exemplary embodiment, the spargers can comprise from 4 to 10 injectors, comprising stainless steel microporous pipes (e.g., having tens or hundreds of holes 1 micron or less in size), which are connected to the ozone supply via, for example, piping. The microporous design allows for even dispersal of ozone throughout the fermentation medium.

The ozone can be applied continuously for a period of time sufficient to achieve sterilization of the fermentation medium within the vessel, for example, from 5 minutes to 48 hours, from 30 minutes to 36 hours, from 60 minutes to 24 hours, or from 90 minutes to 12 hours. The rate of application can depend on the size and production capacity of the ozone generator or ozone machine employed.

Typically, ozone generators require the cooling of ozone in order to prevent premature decomposition of the ozone molecule. Advantageously, in some embodiments, the method can be carried out at ambient temperature, without heating or cooling of the ozone and/or fermentation media components. In some embodiments, however, the ozone can be cooled to, e.g., 5 to 15° C. prior to contact with the fermentation medium using standard ozone cooling methods, if necessary.

In certain embodiments, the fermentation medium is agitated throughout the process of being contacted with the ozone. In some embodiments, this is achieved simply through the use of spargers and the resulting movement of bubbles throughout the medium.

In some embodiments, a mechanical mixing device can be utilized, for example, an internal mixing apparatus comprising an impeller that can be rotatably controlled manually or via a motor. The impeller can help propel liquid between the top and the bottom of the vessel to ensure efficient mixing and gas dispersion throughout.

In some embodiments, external circulation tubing can be used to provide mixing and agitation to the fermentation medium during ozone sterilization. For example, one or more external tubing loops fitting with circulation pumps can transport liquid out of the bottom of the vessel and back into the top of the vessel, and/or vice versa.

In certain embodiments, the vessel comprises an off-gas system, through which excess air and/or ozone escapes. In certain embodiments, the escaped ozone can be recycled into the vessel.

In certain embodiments, the fermentation medium can be tested for contamination after ozone sterilization and prior to inoculation of the medium with a microorganism. If testing reveals that sterilization has not been achieved, then additional ozone treatments can be applied.

In certain embodiments, individual media components are ozone sterilized and then mixed together afterwards.

In some embodiments, the equipment used for fermentation is also sterilized. In some embodiments, ozone can be used for sterilizing the surfaces of the vessel, gaskets, opening, tubing, and other equipment parts. Other sterilizing materials can also be used, such as steam, alcohol or bleach. In some embodiments, the air supplied during aerobic fermentation can be filtered and/or sterilized using ozone or other known methods.

Once sterilization of the fermentation medium is achieved according to the subject methods, the medium can be inoculated with a microorganism of choice to proceed with aerobic fermentation. In certain embodiments, all of the ozone within the fermentation medium has decomposed by the time at which inoculation occurs.

The microbial inoculant can comprise cells and/or propagules of the desired microorganism, which can be prepared using any known cultivation method. The inoculant can be pre-mixed with water and/or a liquid growth medium, if desired.

The microorganisms can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganisms are yeasts and/or fungi. Yeast and fungus species suitable for use according to the current invention include but are not limited to Acaulospora, Acremonium chrysogenum, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Entomophthora, Hanseniaspora (e.g., H. uvarum), Hansenula, Issatchenkia, Kluyveromyces (e.g., K. phaffii), Lentinula spp. (e.g., L. edodes), Meyerozyma (e.g., M. guilliermondii. M. carribica), Monascus purpureus, Mortierella, Mucor (e.g., M. piriformis), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudozyma (e.g., P. aphidis), Rhizopus, Rhodotorula (e.g., R. bogoriensis); Saccharomyces (e.g., S. cerevisiae, S. boulardii, S. torula), Starmerella (e.g., S. bombicola), Torulopsis, Thraustochytrium, Trichoderma (e.g., T. reesei, T. harzianum, T. viride), Ustilago (e.g., U. maydis), Wickerhamiella (e.g., W. domericqiae), Wickerhamomyces (e.g., W. anomalus, W. anomalus NRRL Y-68030), Williopsis (e.g., W. mrakii), Zygosaccharomyces (e.g., Z. bailii), and others.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. amyloliquefaciens NRRL B-67928, B. circulans, B. firmus, B. laterosporus, B. licheniformis, B. megaterium, B. mucilaginosus, B. subtilis, B. subtilis ATCC PTA-123459, B. subtilis NRRL B-68031), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In certain preferred embodiments, the microorganism is a sophorolipid-producing microorganism such as a Starmerella sp. yeast and/or Candida sp. yeast, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214. In certain embodiments, S. bombicola can surprisingly tolerate and/or even thrive in the presence of peroxide in the fermentation medium, which results from the decomposition of ozone.

In certain embodiments, the methods of the subject invention comprise cultivating the microorganism in the vessel to produce a microbial culture, said microbial culture comprising liquid fermentation broth, cells and a fermentation product, e.g., a biosurfactant; extracting the fermentation product from the microbial culture; and, optionally, purifying the fermentation product.

In preferred embodiments, the methods of the subject invention comprise cultivating a sophorolipid-producing yeast in a submerged fermentation reactor to produce a yeast culture, said yeast culture comprising liquid fermentation broth, yeast cells and a mixture of SIP molecules; extracting the SLP from the yeast culture; and, optionally, purifying the SLP.

In some embodiments, the methods comprise testing the extracted and/or purified fermentation product, e.g., biosurfactant, for impurities, color and/or odor.

In preferred embodiments, the microorganism is cultivated via aerobic fermentation. Thus, in preferred embodiments, the method comprises providing oxygenation to the growing culture. One embodiment utilizes slow motion of air to remove low oxygen-containing air and introduce oxygenated air. The oxygenated air may be provided via spargers and/or aeration systems, and further dispersed through agitation mechanisms, including those described previously. In certain embodiments, dissolved oxygen (DO) levels are maintained at about 25% to about 75%, about 30% to about 70%, about 35% to about 65%, about 40% to about 60%, or about 50% of air saturation.

The pH of the culture should be suitable for the microorganism of interest. In some embodiments, the pH is about 2.0 to about 7.0, about 3.0 to about 5.5, about 3.25 to about 4.0, or about 3.5. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH solution. The base solution can be included in the growth medium and/or it can be fed into the fermentation reactor during cultivation to adjust the pH as needed.

In one embodiment, the method of cultivation is carried out at about 5° to about 100° C., about 15° to about 60° C., about 20° to about 45° C., about 22° to about 35° C., or about 24° to about 28° C. In one embodiment, the cultivation may be carried out continuously at a constant temperature. In another embodiment, the cultivation may be subject to changing temperatures.

According to the subject methods, the microorganisms can be cultivated in the fermentation system for a time period sufficient to achieve a desired effect, e.g., production of a desired amount of cell biomass or a desired amount of a metabolite (e.g., SLP). The microbial growth by-product(s) produced by microorganisms may be retained in the microorganisms and/or secreted into the growth medium. The biomass content may be, for example from 5 g/l to 180 g/l or more, from 10 g/l to 150 g/l, or from 20 g/l to 100 g/l.

In certain embodiments, fermentation of the microbial culture occurs for about 40 to 150 hours, or about 48 to 140 hours, or about 72 to 130 hours or about 96 to 120 hours. In certain specific embodiments, fermentation time ranges from 48 to 72 hours, or from 96 to 120 hours.

In some embodiments, the fermentation cycle is ended once the sugar and/or fatty acid concentrations in the medium are exhausted (e.g., at a level of 0% to 0.5%). In some embodiments, the end of the fermentation cycle is determined to be a time point when the microorganisms have begun to consume trace amounts of the biosurfactant.

In certain embodiments, when fermentation is ended, the SLP or other biosurfactant can be extracted from the microbial culture, and, optionally, purified. This can be achieved using methods known in the art.

In some embodiments, the methods further comprise testing the extracted and/or purified biosurfactant for impurities, color and/or odor.

In specific embodiments, color can be tested and compared between fermentation products to determine the effectiveness of substituting ozone sterilization for thermal methods at reducing color and impurities. Browning, in particular, is an indicator of impurities such as Maillard reaction products, which include, for example, melanoidins (brown nitrogenous polymers). In some embodiments, measurement of the color can be conducted using spectral analysis and/or optical turbidity measurements.

In certain embodiments, the methods of the subject invention can be carried out in such a way that minimal-to-zero waste products are produced, thereby reducing the amount of fermentation waste being drained into sewage and wastewater systems, and/or being disposed of in landfills. Furthermore, this can be achieved while increasing the overall biosurfactant production from a single fermentation cycle.

The supernatant that is leftover after extraction can comprise residual cells, broth components, and impurities, such as glucose. In some embodiments, the residual cellular matter can be discarded and/or it can be re-used or recycled, for example, as a soil amendment, a livestock feed supplement, an oil well treatment, and/or a skincare product. The cell biomass can be used directly, or it can be mixed with additives specific for the intended use.

The subject invention provides microbe-based products, as well as uses for these products to achieve beneficial results in many settings including, for example, improved bioremediation, mining, and oil and gas production; food production and processing; waste disposal and treatment; enhanced health of livestock and other animals; and enhanced health and productivity of plants, by applying one or more of the microbe-based products to a desired site. The microbe-based products can be used in formulating, for example, biopesticides, health supplements, pharmaceuticals, remediation agents, cosmetic and/or personal care products, cleaning agents, home care products and/or industrial supplies.

In certain embodiments, the subject invention provides compositions produced according to the subject methods, the compositions comprising a purified SLP having a reduced color and/or impurity content compared with SLP produced with fermentation media sterilized using thermal methods. The purified SLP can be, for example, a lactonic, linear, mono-acetylated lactonic or linear, and/or di-acetylated lactonic or linear sophorolipid. In certain embodiments, the composition comprises more than one purified SLP molecule. In certain embodiments, the SLP can be subjected to further chemical treatments to, for example, produce derivatives of SLP molecules.

Further components can be added to the sophorolipidic compositions as needed for a particular use. The additives can be, for example, buffers, carriers, other microbe-based compositions produced at the same or different facility, viscosity modifiers, preservatives, nutrients for microbe growth, nutrients for plant growth, solvents, tracking agents, pesticides, herbicides, animal feed, food products and other ingredients specific for an intended use.

Cultivation of microbial biosurfactants according to the prior art is a complex, time- and resource-consuming process that requires multiple stages. Advantageously, the methods of the subject invention do not require complicated equipment or high energy consumption, and thus reduce the capital and labor costs of producing microorganisms and their metabolites on a large scale. Additionally, the methods and equipment of the subject invention reduce the capital and labor costs of purifying microbial metabolites on a large scale.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Color Improvement Study

The Gardner Color Scale was used to compare the color of SLP produced using steam-sterilized fermentation medium with SLP produced using ozone-sterilized fermentation medium. Color improvement was observed for both types of SLP for the ozone group.

TABLE 1
Linear SLP Gardner Color

| Steam Average | Ozone Average | Gardner Color Improvement |
|---|---|---|
| 15.6 | 9.75 | 5.85 |

TABLE 2
Lactonic SLP Gardner Color

| Steam Average | Ozone Average | Gardner Color Improvement |
|---|---|---|
| 13.4 | 8.125 | 5.275 |

We claim:

1. A method of producing a fermentation product, the method comprising applying ozone to a fermentation medium; inoculating the fermentation medium with a microorganism; cultivating the microorganism aerobically to produce the fermentation product; obtaining the fermentation product; and, optionally, purifying the fermentation product.

2. The method of claim 1, wherein the fermentation product is a biosurfactant.

3. The method of claim 2, wherein the biosurfactant is a glycolipid selected from sophorolipids, mannosylerythritol lipids, rhamnolipids, and trehalose lipids.

4. The method of claim 3, wherein the biosurfactant is a sophorolipid.

5. The method of claim 1, wherein applying the ozone to the fermentation medium comprises supplying the ozone directly to the inside of a vessel in which the fermentation occurs using an ozone generator or ozone machine.

6. The method of claim 5, wherein the ozone generator or ozone machine is coupled directly to air spargers situated in the vessel.

7. The method of claim 1, wherein the ozone is applied at room temperature.

8. The method of claim 1, wherein the ozone is applied continuously for a period of time from 5 minutes to 48 hours.

9. The method of claim 1, wherein the fermentation medium is mixed continuously throughout application of the ozone.

10. The method of claim 1, wherein prior to inoculation of the fermentation medium with the microorganism, the medium is sampled and tested for contamination.

11. The method of claim 1, wherein the microorganism is Starmerella (Candida) bombicola, Candida apicola, Candida batistae, Candida floricola, Candida riodocensis, Candida stellate and/or Candida kuoi.

12. The method of claim 1, wherein the fermentation product has a reduced color compared with the same product produced using fermentation media that are sterilized using thermal methods.

13. The method of claim 1, wherein the media has reduced Maillard reaction products compared with media that is sterilized using thermal methods.

14. The method of claim 1, wherein the amount of the fermentation product that is produced is greater than the amount of said product produced using fermentation media sterilized using thermal methods.

15. A method of sterilizing a fermentation medium without the use of heat, the method comprising applying ozone to the fermentation medium prior to inoculation of the medium with a microorganism.
